(19) United States
(12) Patent Application Publication
(10) Pub. No.: US 2018/0098925 A1
(43) Pub. Date: Apr. 12, 2018

(54) AGENT AND METHOD FOR LIGHTENING KERATIN-CONTAINING FIBERS

(71) Applicant: Henkel AG & Co. KGaA, Duesseldorf (DE)

(72) Inventors: Udo Erkens, Willich (DE); Burkhard Mueller, Duesseldorf (DE)

(73) Assignee: Henkel AG & Co. KGaA, Duesseldorf (DE)

(21) Appl. No.: 15/730,778

(22) Filed: Oct. 12, 2017

(30) Foreign Application Priority Data
Oct. 12, 2016 (DE) 10 2016 219 863.7

(51) Int. Cl. A61K 8/22; A61K 8/25; A61K 8/02; A61Q 5/08; A61Q 5/10

(52) U.S. Cl. CPC A61K 8/22; A61K 8/25; A61K 2800/882; A61Q 5/08; A61Q 5/10; A61K 8/022

(57) ABSTRACT

Cosmetic agent for lightening keratin fibers, containing at least one packaged preparation (A), wherein the preparation (A), based on its total weight, contains
a) from about 10 to about 90% by weight of peroxydisulfate(s)
b) from about 10 to about 50% by weight of sodium silicate
and the preparation (A) is packaged in a water-soluble film which, based on its total weight, includes at least 60% by weight of a polymer mixture which has a multi-modal molecular weight distribution, exemplified in that the sodium silicate comprises sodium disilicate and methods for the use of this agent.

### CROSS-REFERENCE TO RELATED APPLICATION

This application claims priority to German Patent Application No. 10 2016 219 863.7, filed Oct. 12, 2016, which is incorporated herein by reference in its entirety.

### TECHNICAL FIELD

The invention relates to a cosmetic agent comprising a cosmetic preparation packaged in a water-soluble and/or water-dispersible coating and a method for the bleaching of keratin-containing fibers.

### BACKGROUND

In the field of decorative cosmetics, in particular bleaching or dyeing hair cosmetics, there is a great need for effective and, at the same time, simple and safe manageable products. In particular in the field of hair cosmetics, the consumer is provided with bleaching and hair dying systems which are extremely effective, but, with improper handling, can lead to irritation or even in extreme cases, to the triggering of allergies, for example, when there is contact with skin parts or eyes. There is therefore a great need to ensure the safe handling of such cosmetic formulations and, in addition, to provide the consumer with a packaging system that is easy to dose by hand which also allows a mixing or an assembly of the required components on site. An important point, in particular with respect to bleaching or dyeing hair cosmetics, is therefore the avoidance of product dusts.

In the literature, there are first approaches to solving the technical problems described above. Thus, the German patent application DE 196 13 941 A1 describes a method for the production of non-dusting powdery bleaching agents. The bleaching agents have at least one peroxide compound, which is mixed with suitable thickening agents and subsequently packaged by portions in water-soluble pouches for transport and further processing.

European patent EP 493 392 B1 discloses hair bleaching and dyeing agents which are incorporated into polyvinyl alcohol packagings to reduce the irritation caused by powder dust.

The European patent application EP 1 510 529 A1 describes the preparation of multi-modal dispersions of vinyl alcohol/vinyl acetate copolymers.

The international patent application WO 2004/082818 A2 describes a mixing device for mixing a liquid with a filled film pouch which is soluble in this liquid. The film pouch may, for example, contain bleaching or dyeing agents.

International patent application WO 2016/074854 A1 describes cosmetic agents for whitening keratin fibers, which are packaged in a water-soluble film which includes at least in part a polymer mixture having a multi-modal molecular weight distribution.

Although the portioned cosmetic formulations disclosed in the prior art offer improved handleability and a reduction in the dust contamination of the packaged cosmetic preparations, the portions packaged in water-soluble film systems have the disadvantage that they dissolve only slowly in water or are chemically unstable.

### BRIEF SUMMARY

Cosmetic agents and methods for the color change of keratin fibers are provided. In an embodiment, a cosmetic agent for lightening keratin fibers includes at least one packaged preparation (A). The preparation (A), based on its total weight, includes from about 10 to about 90% by weight of peroxodisulfate(s) and from about 10 to about 50% by weight of sodium silicate, wherein the sodium silicate includes sodium disilicate. The preparation (A) is packaged in a water-soluble film which, based on its total weight, includes at least about 60% by weight of a polymer mixture which has a multi-modal molecular weight distribution.

In another embodiment, a cosmetic agent includes at least one packaged preparation (A), wherein the preparation (A), based on its total weight, includes from about 10 to about 90% by weight of peroxodisulfate(s) and from about 10 to about 50% by weight of sodium disilicate. The preparation (A) is packaged in a water-soluble film which, based on its total weight, includes at least about 60% by weight of a polymer mixture which has a multi-modal molecular weight distribution.

In another embodiment, a method for the color change of keratin fibers is provided, in the course of which at least one packaged preparation (A) and a preparation (B) are mixed into an application mixture in which the preparation (A) packaged in a water-soluble film is introduced into the free-flowing preparation (B) by dissolving the water-soluble film. The preparation (A), based on its own weight includes from about 10 to about 90% by weight of peroxodisulfate(s) and from about 10 to about 50% by weight of sodium silicate. The sodium silicate includes sodium disilicate. Preparation (A) is packaged in a water-soluble film which includes, based on its own weight, at least about 60% by weight, of a polymer mixture that has a multi-modal molecular weight distribution. Preparation (B) comprises water and at least one oxidation agent. The resulting application mixture is applied to the keratin fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure is to provide portioned cosmetic preparations for bleaching keratin fibers which are storage-stable, simple, safe and handled quickly, which can thus be handled, for example, without dust development and provide the consumer with a ready-for-use application mixture in a short time.

It has been found that the aforementioned problems can be solved by combining specific bleach compositions with specific packaging agents. This combination increases the storage stability of the packaged compositions, which not only have a reduced dust development in the application, but also dissolve surprisingly rapidly and residue-free.

The agents and methods as contemplated herein and some of their preferred embodiments are exemplified by the following points:

1. Cosmetic agent for lightening keratin fibers, comprising at least one packaged preparation (A), wherein the preparation (A), based on its total weight, contains
a) from about 10 to about 90% by weight of peroxydisulfate(s)
b) from about 10 to about 50% by weight of sodium silicate
and the preparation (A) is packaged in a water-soluble film which, based on its total weight, comprises at least about 60% by weight of a polymer mixture which has a multi-modal molecular weight distribution, exemplified in that the sodium silicate comprises sodium disilicate.
2. Cosmetic agent according to point 1, exemplified in that the composition (A), based on its total weight, includes from about 20 to about 80% by weight, preferably from about 30 to about 60% by weight of peroxydisulfate(s).
3. Cosmetic agent according to one of the preceding points, exemplified in that the peroxydisulfate is selected from the group of alkali metal and ammonium peroxydisulfates, in particular sodium peroxydisulfate, potassium peroxydisulfate, ammonium peroxydisulfate and mixtures thereof.
4. Cosmetic agent according to one of the preceding points, exemplified in that the peroxydisulfate is selected from the group of alkali metal and ammonium peroxydisulfates, in particular sodium peroxydisulfate, potassium peroxydisulfate, ammonium peroxydisulfate and mixtures thereof.
5. Cosmetic agent according to one of the preceding points, exemplified in that the weight ratio of potassium peroxydisulfate contained in the agent to the total amount of peroxydisulfates contained in the agent is at least about 0.5, preferably at least about 0.7, more preferably at least about 0.9.
6. Cosmetic agent according to one of the preceding points, exemplified in that the sodium silicate, based on its total weight, includes at least about 20% by weight, preferably at least about 50% by weight, more preferably at least about 80% by weight and in particular completely of sodium disilicate.
7. Cosmetic agent according to one of the preceding points, including at least one packaged preparation (A), wherein the preparation (A), based on its total weight, contains
a) from about 10 to about 90% by weight of peroxydisulfate(s)
b) from about 10 to about 50% by weight of sodium disilicate
and the preparation (A) is packaged in a water-soluble film which, based on its total weight, includes at least about 60% by weight of a polymer mixture which has a multi-modal molecular weight distribution.
8. Cosmetic agent according to one of the preceding points, exemplified in that the preparation (A), based on its total weight contains,
c) from about 5 to about 30% by weight, preferably from about 10 to about 20% by weight, of carbonate(s).
9. Cosmetic agent according to one of the preceding points, exemplified in that the preparation (A), based on its total weight contains,
c) from about 5 to about 30% by weight, preferably from about 10 to about 20% by weight, of magnesium carbonate.
10. Cosmetic agent according to one of the preceding points, exemplified in that the water-soluble film, based on its total weight, includes at least about 70% by weight, preferably at least about 80% by weight, more preferably at least about 90% by weight and in particular at least about 95% by weight of a polymer mixture which has a multi-modal molecular weight distribution.
11. Cosmetic agent according to one of the preceding points, wherein the polymer mixture includes, based on its total weight, at least about 60% by weight, preferably at least about 80% by weight, more preferably at least about 90% by weight, particularly preferably at least about 95% by weight, of a mixture comprising
a1) water-soluble vinyl alcohol/vinyl acetate copolymer a1) and
a2) at least one water-soluble vinyl alcohol/vinyl acetate copolymer a2) which is different from the water-soluble vinyl alcohol/vinyl acetate copolymer a1)
12. Cosmetic agent according to one of the preceding points, exemplified in that the polymer mixture includes, based on its total weight, at least about 60% by weight, preferably at least about 80% by weight, more preferably at least about 90% by weight, particularly preferably at least about 95% by weight, of a mixture comprising
a1) water-soluble vinyl alcohol/vinyl acetate copolymer a1) and
a2) at least one optionally modified water-soluble polysaccharide, preferably at least one water-soluble polysaccharide from the group of methylcellulose, carboxymethylcellulose, ethylcellulose, hydroxyethylcellulose, hydroxypropylcellulose, hydroxypropylmethylcellulose, dextrin and hydroxypropylstarch, more preferably at least one water-soluble polysaccharide from the group of hydroxypropyl starches
13. Cosmetic agent according to one of the preceding points, exemplified in that the polymer mixture has a polydispersity index above about 2.2, preferably above about 3.0, in particular above about 4.2.
14. Cosmetic agent according to one of the preceding points, exemplified in that the vinyl alcohol/vinyl acetate copolymer a1) has a polydispersity index of between about 1.8 and about 2.3.
15. A cosmetic agent according to one of the preceding points, exemplified in that the vinyl alcohol/vinyl acetate copolymer a1) has a degree of hydrolysis between about 84% and about 90%, preferably between about 85% and about 89%, and in particular between about 86% and about 88%.
16. Cosmetic agent according to one of the preceding points, exemplified in that the vinyl alcohol/vinyl acetate copolymer a1) has a viscosity (20° C., 4% by weight solution in water, measured with a Brookfield LV viscometer with UL adapter) of between about 12 cP and about 20 cP, preferably between about 14 cP and about 19 cP, and in particular between about 16 cP and about 18 cP.
17. Cosmetic agent according to one of the preceding points, exemplified in that the vinyl alcohol/vinyl acetate copolymer a2) has a viscosity (20° C., 4% by weight solution in water, measured with a Brookfield LV viscometer with UL adapter) of between about 20 cP and about 30 cP, preferably between about 20 cP and about 28 cP, and in particular between about 20 cP and about 25 cP.
18. Cosmetic agent according to one of the preceding points, exemplified in that the water-soluble film, based on its total weight, has a water content of from about 3.0 to about 12% by weight, preferably of from about 4.0 to about 10% by weight.
19. Cosmetic agent according to one of the preceding points, exemplified in that the water-soluble film has a thickness of from about 0.01 to about 0.1 mm, preferably of from about 0.01 to about 0.08 mm and in particular of from about 0.02 to about 0.06 mm.
20. Cosmetic agent according to one of the preceding points, exemplified in that the cosmetic agent further comprises a flowable preparation (B) which contains water and at least one oxidizing agent.
21. Cosmetic agent according to one of the preceding points, exemplified in that the cosmetic agent further comprises a flowable preparation (B) which, based on its weight, contains from about 50 to about 98% by weight, preferably from about 60 to about 95% by weight, more preferably from about 80 to about 95% by weight of water and from about 0.5 to about 20% by weight, preferably from about 1 to about 15% by weight, particularly preferably from about 2 to about 12% by weight of hydrogen peroxide, calculated as 100% H₂O₂.
22. Cosmetic agent according to one of points 20 or 21, exemplified in that the weight ratio of the preparation (A) to the preparation (B) is from about 1:1 to about 1:10, preferably from about 1:1 to about 1:5, and in particular from about 1:1 to about 1:2.
23. Method for the color change of keratin fibers, in the course of which
i) at least one packaged preparation (A) and a preparation (B) are mixed into an application mixture in which the preparation (A) packaged in a water-soluble film is introduced into the free-flowing preparation (B) by dissolving the water-soluble film, wherein the preparation (A), based on its own weight, contains
a) from about 10 to about 90% by weight of peroxydisulfate(s)
b) from about 10 to about 50% by weight of sodium silicate
and is packaged in a water-soluble film which includes, based on its own weight, at least about 60% by weight, of a polymer mixture that has a multi-modal molecular weight distribution, and wherein the preparation (B) contains water and at least one oxidation agent, exemplified in that the sodium silicate includes sodium disilicate;
ii) the resulting application mixture is applied to the keratin fibers.

The preparation (A) encompassed by the agent as contemplated herein contains, based on its own weight, from about 10 to about 90% by weight of peroxydisulfate(s) as its first essential constituent. Preference is given to the use of from about 20 to about 80% by weight and in particular from about 30 to about 60% by weight of peroxydisulfate(s).

Preferred peroxydisulfates to be used are the alkali metal and ammonium peroxydisulfates, in particular sodium peroxydisulfate, potassium peroxydisulfate, ammonium peroxydisulfate and mixtures thereof. The weight ratios of the different peroxidisulfates are, in principle, freely selectable, but can also be determined within predetermined limits. For example, the amount of potassium peroxydisulfate can always be kept higher than the amount of sodium and ammonium peroxydisulfate which may be used. This is illustrated, for example, in an embodiment of cosmetic agents as contemplated herein, in which the preparation (A), based on its weight, contains from about 10 to about 90% by weight, preferably from about 20 to about 80% by weight, more preferably from about 30 to about 60% by weight of potassium peroxydisulfate and the weight ratio of potassium peroxydisulfate contained in the agent to the total amount of peroxydisulfates contained in the agent is at least about 0.5, preferably at least about 0.7, more preferably at least about 0.9.

As its second essential component, the preparation (A) contains from about 10 to about 50% by weight of sodium silicate, which in turn must comprise sodium disilicate.

Sodium orthoslicate, sodium metasilicate, sodium disilicate and sodium trisilicate can be distinguished in the sodium silicate used in preparation (A). Surprisingly, it has proved to be advantageous for the storage stability and the dissolution properties of the cosmetic agents to use a sodium disilicate as sodium silicate b).

Preferred cosmetic agents are exemplified in that the sodium silicate used includes, based on its total weight, at least about 20% by weight, preferably at least about 50% by weight, more preferably at least about 80% by weight and in particular completely, of sodium disilicate.

Accordingly, particularly preferred are cosmetic agents containing at least one packaged preparation (A), wherein the preparation (A), based on its total weight, contains

a) from about 10 to about 90% by weight of peroxydisulfate(s)
b) from about 10 to about 50% by weight of sodium disilicate
and the preparation (A) is packaged in a water-soluble film which, based on its total weight, includes at least about 60% by weight of a polymer mixture which has a multi-modal molecular weight distribution.

In addition to the above-described peroxydisulfates and sodium silicates, preferred preparations (A) contain at least one additional alkali source, in particular carbonates. The weight fraction of the carbonate in the total weight of the preparation (A) is preferably from about 5 to about 30% by weight and more preferably from about 10 to about 20% by weight. Particular preference is given to the use of magnesium carbonate.

In summary, cosmetic agents are preferred which contain at least one packaged preparation (A), wherein the preparation (A), based on its total weight, contains

a) from about 10 to about 90% by weight of peroxydisulfate(s)
b) from about 10 to about 50% by weight of sodium disilicate
c) from about 5 to about 30% by weight of magnesium carbonate
and the preparation (A) is packaged in a water-soluble film which, based on its total weight, includes at least about 60% by weight of a polymer mixture which has a multi-modal molecular weight distribution.

The preparation (A) is preferably present in solid form, for example, in the form of a powder, a granulate or a compressed body, for example, in the form of a tablet. Preferred cosmetic preparations (A) are in powder form.

The preparation (A) encompassed by the cosmetic agent as contemplated herein is packaged in a water-soluble film. The molecular weight distribution of the polymer mixture contained in this water-soluble film is multi-modal. In other words, the density of the frequency distribution of the molecular weight has at least two modes (maxima), for example two, three, four, five or more modes. Particular preference is given to a bimodal molecular weight distribution, since, as described at the outset, on the one hand this has a very advantageous effect on the product properties of cosmetic agents as contemplated herein, this is easier to realize on the other hand than a tri- or multi-modal frequency distribution.

The preferred bimodal molecular weight distribution may be symmetric or asymmetric.

In a preferred multi-modal, preferably bimodal, molecular weight distribution, the molecular weights differ by at least two of the modes relative to the smallest molecular weight attributable to a mode by from about 5% to about 120%, preferably by from about 10% to about 90% and in particular by from about 20% to about 60%

In a further preferred multi-modal, preferably bimodal, molecular weight distribution, the frequency of the two-mode minimum differs from the frequency of the least of these two modes (least-frequent mode) by from about 5% to about 80%, preferably from about 10% to about 60%, in particular from about 20% to about 40%, in each case based on the frequency of the smallest of the two modes.

For the application properties of agent as contemplated herein, in particular the rapid and residue-free preparation of the hair cosmetic application mixture, it has proved to be advantageous when the water-soluble film, based on its own weight, includes at least about 70% by weight, preferably at least about 80% by weight, more preferably at least about 90% by weight, and in particular at least about 95% by weight, of a polymer mixture which has a multi-modal molecular weight distribution. Again, a bimodal molecular weight distribution is preferred.

Polymer mixtures which have a polydispersity index above about 2.2, preferably above about 3.0, and in particular above about 4.2, have proved to be advantageous for the product properties. The ratio of weight-average and number-average molecular weight in this case is referred to as the polydispersity index.

The weighting agent or the weight-average molecular weight (Mᵢₙᵢₜ) is defined as

Mᵢₙᵢₜ=ΣnᵢMᵢ²/ΣnᵢMᵢ

with Mᵢₙᵢₜ=weight-average molecular weight, Nᵢ=number of macromolecules in the sample with exactly i repeating units and Mᵢ=molecular weight i.
The average weight is obtained by methods that take into account the size and shape of a molecule in solution, e.g., static light scattering, X-ray scattering and sedimentation equilibrium measurements.

The number-average or the number-average molecular weight (Mₙ) is defined as

Mₙ=ΣnᵢMᵢ²/ΣnᵢMᵢ

with Mᵢₙᵢₜ=weight-average molecular weight, Nᵢ=number of macromolecules in the sample with exactly i repeating units and Mᵢ=molecular weight i.
The number-average can be determined by colligative methods such as, for example, cryoscopy, membrane or vapor pressure osmometry and, if the number of end groups per molecule is known, by end group determination.

Water-soluble films which do not completely include the polymer mixture with the multi-modal molecular weight distribution can contain additional active ingredients or fillers, but also solvents, in particular water, as further ingredients.

The group of the further active ingredients includes, for example, hair-cosmetically active components as well as materials which protect the ingredients of preparation (A) enclosed by the film material from decomposition or deactivation by light irradiation. Antioxidants, UV absorbers and fluorescent dyes have proved to be particularly suitable here.

The water-soluble film, based on its total weight, preferably has a water content of from about 3.0 to about 12% by weight, preferably of from about 4.0 to about 10% by weight.

The thickness of the water-soluble film used for packaging the preparation (A) is preferably from about 0.01 to about 0.1 mm, more preferably from about 0.01 to about 0.08 mm and in particular from about 0.02 to about 0.06 mm.

The water-soluble film into which preparation (A) is packaged may comprise one or more structurally different water-soluble polymers. Polymers from the group (optionally acetalized) of polyvinyl alcohols (PVAL), polyvinylpyrrolidones, polyethylene oxides, gelatin and cellulose are particularly suitable as water-soluble polymer(s).

In a first preferred embodiment, the polymer mixture having the multi-modal, preferably bimodal, molecular weight distribution comprises two vinyl acetate/vinyl alcohol copolymers. Preferred cosmetic agents are therefore exemplified in that the polymer mixture comprises, based on its total weight, at least about 60% by weight, preferably at least about 80% by weight, preferably at least about 90% by weight, particularly preferably at least about 95% by weight of a mixture comprising

a1) water-soluble vinyl alcohol/vinyl acetate copolymer a1) and
a2) at least one water-soluble vinyl alcohol/vinyl acetate copolymer a2) which is different from the water-soluble vinyl alcohol/vinyl acetate copolymer a1)

In the aforementioned preferred embodiment based on two water-soluble vinyl alcohol/vinyl acetate copolymers, the polymer mixture preferably has a polydispersity index above about 2.2, preferably above about 3.0 and in particular above about 4.2, while the polydispersity index of the vinyl alcohol/vinyl acetate copolymer a1) is preferably is between about 1.8 and about 2.3.

Particularly advantageous product properties are achieved by vinyl alcohol/vinyl acetate copolymers a1) having a degree of hydrolysis between about 84% and about 90%, preferably between about 85% and about 89% and in particular between about 86% and about 88%. In other words, corresponding copolymers a1) have a residual content of acetyl groups of between about 10% and about 16%, preferably between about 11% and about 15%, and in particular between about 12% and about 14%.

In addition to the polydispersity index and the degree of hydrolysis, the viscosity of aqueous solutions of the vinyl alcohol/vinyl acetate copolymers has proved to be a characteristic feature of particularly advantageous copolymers. Preferred cosmetic agents are therefore exemplified in that the vinyl alcohol/vinyl acetate copolymer a1) has a viscosity (20° C., 4% by weight solution in water, measured with a Brookfield LV viscometer with UL adapter) of between about 12 cP and about 20 cP, preferably between about 14 cP and about 19 cP, and in particular between about 16 cP and about 18 cP.

On the other hand, the viscosity (20° C., 4% by weight solution in water, measured with a Brookfield LV viscosimeter with UL adapter) of the vinyl alcohol/vinyl acetate copolymer a2) is preferably between about 20 cP and about 30 cP, preferably between about 20 cP and about 28 cP and in particular between about 20 cP and about 25 cP.

In addition to the above-described combination of two vinyl alcohol/vinyl acetate copolymers, there are further preferred polymer combinations having properties which are advantageous in view of the abovementioned technical tasks. In an alternative preferred embodiment of cosmetic agents as contemplated herein, the polymer mixture, based on its own weight, includes at least about 60% by weight, thus preferably at least about 80% by weight, more preferably at least about 90% by weight, particularly preferably at least about 95% by weight of a mixture comprising

a1) water-soluble vinyl alcohol/vinyl acetate copolymer a1) and
a2) at least one optionally modified water-soluble polysaccharide, preferably at least one water-soluble polysaccharide from the group of methylcellulose, carboxymethylcellulose, ethylcellulose, hydroxyethylcellulose, hydroxypropylcellulose, hydroxypropylmethylcellulose, dextrin and hydroxypropylstarch, more preferably at least one water-soluble polysaccharide from the group of hydroxypropyl starches.

The polydispersity index of the abovementioned polymer mixtures of vinyl alcohol/vinyl acetate copolymers and polysaccharide is again preferably above about 2.2, more preferably above about 3.0 and in particular above about 4.2, whereas the vinyl alcohol/vinyl acetate copolymer a1) preferably has a polydispersity index of between about 1.8 and about 2.3.

If the vinyl alcohol/vinyl acetate copolymer a1) is combined with a polysaccharide, the vinyl alcohol/vinyl acetate copolymer a1) preferably has a degree of hydrolysis between about 84% and about 90%, preferably between about 85% and about 89%, and in particular between about 86% and about 88%. The viscosity (20° C., 4% strength by weight solution in water, measured with a Brookfield LV viscosimeter with UL adapter) of the vinyl alcohol/vinyl acetate copolymer a1) is preferably between about 12 cP and about 20 cP, more preferably between about 14 cP and about 19 CP and in particular between about 16 cP and about 18 cP.

The preparation (A) is preferably mixed with the aqueous preparation (B) before use on the hair and dissolved in it. The distinguishing constituent of the aqueous preparation (B) is at least one oxidizing agent. The weight ratio of the preparation (A) to the preparation (B) is preferably from about 1:1 to about 1:10, more preferably from about 1:1 to about 1:5 and in particular from about 1:1 to about 1:2.

In a preferred embodiment, the preparation (B) is an aqueous hydrogen peroxide solution. Preference is given to a preparation (B) which, based on its weight, contains from about 50 to about 98% by weight, preferably from about 60 to about 95% by weight, more preferably from about 80 to about 95% by weight of water and from about 0.5 to about 20% by weight, preferably from about 1 to about 15% by weight, particularly preferably from about 2 to about 12% by weight, of hydrogen peroxide, calculated as 100% H₂O₂.

As a further constituent, the preparation (B) preferably contains emulsifiers or surface-active agents.

A first group more preferred are the anionic surfactants. Anionic surfactants for the purposes of the present disclosure are all anionic surface-active substances suitable for use on the human body. These are exemplified by a water-solubilizing, anionic group such as, for example, a carboxylate, sulfate, sulfonate or phosphate group and a lipophilic alkyl group having from 8 to 30 carbon atoms. In addition, glycol or polyglycol ether groups, ester, ether and amide groups and hydroxyl groups may be contained in the molecule. Examples of such anionic surfactants are linear and branched fatty acids having from about 8 to about 30 carbon atoms, in each case in the form of the sodium, potassium and ammonium mono-, di- and trialkanolammonium salts having from 2 to 4 carbon atoms in the alkanol group (soap); ether carboxylic acids, in particular of the formula RO(CH₂CH₂O)ₓCH₂COOH, in which R is a linear alkyl group having from 8 to 30 carbon atoms and x=0 or 1 to 16; acyl sarcosides; acyltaurides; acyl isethionates; sulfosuccinic acid mono- and dialkyl esters, and sulfosuccinic acid monoalkyl polyoxyethyl esters; linear alkane sulfonates; linear olefinsulfonates; sulfonates of unsaturated fatty acids; sulfofatty acid methyl ester of fatty acids; alkyl sulfates and alkyl ether sulfates, in particular of the formula RO(CH₂CH₂O)ₓSO₃H, in which R is a linear alkyl group having from 8 to 30 carbon atoms and x stands for 0 or a number from about 1 to about 12; mixtures of surface-active hydroxysulfonates; sulfated hydroxyalkylpolyethylene and/or hydroxyalkylenepropylene glycol ethers; esters of tartaric acid and citric acid with alcohols; alkyl and/or alkenyl ether phosphates of the formula RO(C₂H₄O)ₓP(═O)(OH)(OR′), wherein R is an aliphatic, optionally unsaturated hydrocarbon radical having from 8 to 30 carbon atoms, R′ stands for hydrogen, a radical (CH₂CH₂O)_{y}R, and x and y each independently stand for a number from about 1 to about 10; sulfated fatty acid alkylene glycol esters of the formula RC(O)O(alkO)ₙSO₃H, in which R stands for a linear or branched, aliphatic, saturated and/or unsaturated alkyl radical having from 6 to 22 carbon atoms, alk stands for CH₂CH₂, CHCH₃CH₂ and/or CH₂CHCH₃, and n stands for a number from about 0.5 to about 5; and monoglyceride sulfates and monoglyceride ether sulfates.

Cosmetic agents preferred as contemplated herein are exemplified in that they additionally contain at least one anionic surfactant. Preferred anionic surfactants are fatty acids, alkyl sulfates, alkyl ether sulfates and ether carboxylic acids having from 10 to 20 carbon atoms in the alkyl group and up to 16 glycol ether groups in the molecule. Particular preference is given to C₈-C₂₀ alkyl sulfates, in particular sodium cetaryl sulfate and sodium lauryl sulfate, and C₈-C₂₀ alkyl ether sulfates having from 2 to 12, preferably 2 to 4, ethylene oxide groups, in particular sodium lauryl ether sulfate (INCI: sodium laureth sulfate). The weight fraction of the anionic surfactant in the total weight of the preparation (B) is preferably from about 0.1 to about 8.0% by weight, preferably from about 0.1 to about 4.0% by weight and in particular from about 0.1 to about 2.0% by weight.

Preferred emulsifiers are furthermore PEG derivatives of hydrogenated castor oil, which, for example, are available under the designation PEG-30 hydrogenated castor oil, e.g., PEG-33 hydrogenated castor oil, PEG-35 hydrogenated castor oil, PEG-36 hydrogenated castor oil or PEG-40 hydrogenated castor oil. As contemplated herein, the use of PEG-40 hydrogenated castor oil is preferred. These are preferably contained in an amount of from about 0.05 to about 1.5% by weight, more preferably from about 0.1 to about 1.0% by weight, even more preferably from about 0.2 to about 0.8% by weight or from about 0.3 to about 0.6% by weight.

The above-described preferred emulsifiers and surface-active agents, to a surprising extent, not only improve the cosmetic performance of cosmetic agents as contemplated herein but also their dissolution and application properties.

In order to be able to apply the application mixture from preparation (A) and (B) cleanly and locally, a higher viscosity of the composition has proved to be advantageous. For this purpose, it is advantageous if the composition is not a paste, a viscous cream or a concentrated gel but rather has a sufficient flowability. Furthermore, the ready-to-use agent must have rheological properties which allow application to the fibers to be bleached but at the same time prevent the agent from running or flowing out from the site of action during the period of use. The application mixtures therefore preferably have a viscosity from about 5 to about 100 Pa·s, preferably from about 10 to about 50 Pa·s, in particular from about 10 to about 20 Pa·s, and particularly preferably from about 10 to about 16 Pa·s (Brookfield, 22° C., spindle #5, 4 rpm). For this purpose, preferred preparations (A) contain at least one thickening agent and/or at least one gelling agent. Corresponding methods as contemplated herein, in which the preparation (A), additionally contains at least one thickening agent and/or at least one gelling agent, are preferred as contemplated herein. Suitable thickening agents or gelling agents are inorganic as well as organic substances.

The thickening agent can be selected, for example, from the polymeric thickening agents known under the following INCI designations: acrylamide copolymer, acrylamide/sodium acrylate copolymer, acrylamide/sodium acryloyldimethyltaurate copolymer, acrylates/acetoacetoxyethyl methacrylate copolymer, acrylates/beheneth-25 methacrylate copolymer, acrylates/C10-30 alkyl acrylate crosspolymer, acrylates/ceteth-20 itaconate copolymer, acrylates/ceteth-20 methacrylate copolymer, acrylates/laureth-25 methacrylate copolymer, acrylate/palmeth-25 acrylate copolymer, acrylate/palmeth-25 itaconate copolymer, acrylate/steareth-50 acrylate copolymer, acrylates/steareth-20 itaconate copolymer, acrylate/steareth-20 methacrylate copolymer, acrylate/stearyl methacrylate copolymer, acrylate/vinyl isodecanoate crosspolymer, acrylic acid/acrylonitrogen copolymer, agar, agarose, alcaligenes polysaccharides, algin, alginic acid, ammonium acrylates/acrylonitrogen copolymer, ammonium acrylate copolymer, ammonium acryloyldimethyltaurate/vinylformamide copolymer, ammonium acryloyldimethyltaurate/VP copolymer, ammonium alginates, ammonium polyacryloyldimethyl taurate, amylopectin, ascorbyl methylsilanol pectinate, astragalus gummifer gum, attapulgite, avena sativa (oat) kernel flour, bentonite, butoxy chitosan, caesalpinia spinosa gum, calcium alginate, calcium carboxymethyl cellulose, calcium carrageenan, calcium potassium carbomer, calcium starch octenylsuccinate, C20-40 alkyl stearates, carbomer, carboxybutyl chitosan, carboxymethyl chitin, carboxymethyl chitosan, carboxymethyl dextran, carboxymethyl hydroxyethylcellulose, carboxymethyl hydroxypropyl guar, cellulose acetate propionate carboxylates, cellulose gum, ceratonia siliqua gum, cetyl hydroxyethylcellulose, cholesterol/hdi/pullulan copolymer, cholesteryl hexyl dicarbamate pullulan, cyamopsis tetragonoloba (guar) gum, diglycol/CHDM/isophthalates/SIP copolymer, dihydrogenated tallow benzylmonium hectorite, dimethicone crosspolymer-2, dimethicone propyl PG betaine, DMAPA acrylates/acrylic acid/acrylonitrogen copolymer, ethylene/sodium acrylate copolymer, gelatin, gellan gum, glyceryl alginates, glycine soybean (soybean) flour, guar hydroxypropyltrimonium chloride, hectorite, hydrated silica, hydrogenated potato starch, hydroxybutyl methylcellulose, hydroxyethyl acrylate/sodium acryloyldimethyl taurate copolymer, hydroxyethylcellulose, hydroxyethyl chitosan, hydroxyethyl ethylcellulose, hydroxypropylcellulose, hydroxypropyl chitosan, hydroxypropyl ethyleneediamine carbomer, hydroxypropyl guar, hydroxypropyl methylcellulose, hydroxypropyl methylcellulose stearoxy ether, hydroxystearamides MEA, isobutylene/sodium maleate copolymer, lithium magnesium silicate, lithium magnesium sodium silicate, macrocystis pyrifera (kelp), magnesium alginate, magnesium aluminum silicate, magnesium silicate, magnesium trisilicate, methoxy PEG-22/dodecyl glycol copolymer, methylcellulose, methyl ethylcellulose, methylhydroxyethylcellulose, microcrystalline cellulose, montmorillonites, moroccan lava clay, nano gum, nonoxynyl hydroxyethylcellulose, octadecenes/MA copolymer, pectin, PEG-800, PEG-crosspolymer, PEG-150/decyl alcohol/SMDI copolymer, PEG-175 diisostearates, PEG-190 distearates, PEG-15 glyceryl tristearates, PEG-140 glyceryl tristearates, PEG-240/HDI copolymer bis-decyltetradeceth-20 ether, PEG-100/IPDI copolymer, PEG-180/laureth-50/TMMG copolymer, PEG-10/lauryl dimethicone crosspolymer, PEG-15/lauryl dimethicone crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 methyl glucose trioleate, PEG-180/octoxynol-40/TMMG copolymer, PEG-150 pentaerythrityl tetrastearate, PEG-4 rapeseedamides, PEG-150/stearyl alcohol/SMDI copolymer, polyacrylate-3, polyacrylic acid, polycyclopentadienes, polyether-1, polyethylene/isopropyl maleate/MA copolyol, polymethacrylic acid, polyquaternium-52, polyvinyl alcohol, potassium alginate, potassium aluminum polyacrylate, potassium carbomer, potassium carrageenan, potassium polyacrylate, potato starch modified, PPG-14 laureth-60 hexyl dicarbamate, PPG-14 laureth-60 isophoryl dicarbamate, PPG-14 palmeth-60 hexyl dicarbamate, propylene glycol alginate, PVP/decene copolymer, PVP montmorillonite, rhizobian gum, ricinoleic acid/adipic acid/AEEA copolymer, sclerotium gum, sodium acrylates/acryloyldimethyl taurate copolymer, sodium acrylate/acrolein copolymer, sodium acrylate/acrylonitrogen copolymer, sodium acrylate copolymer, sodium acrylates/vinyl isodecanoate crosspolymer, sodium acrylate/vinyl alcohol copolymer, sodium carbomer, sodium carboxymethyl chitin, sodium carboxymethyl dextran, sodium carboxymethyl beta glucan, sodium carboxymethyl starch, sodium carrageenan, sodium cellulose sulfates, sodium cyclodextrin sulfates, sodium hydroxypropyl starch phosphates, sodium isooctylene/MA copolymer, sodium magnesium fluorosilicates, sodium polyacrylates, sodium polyacrylates starch, sodium polyacryloyldimethyl taurates, sodium polymethacrylates, sodium polystyrene sulfonates, sodium silicoaluminate, sodium starch octenylsuccinate, sodium stearoxy PG-hydroxyethylcellulose sulfonate, sodium styrene/acrylate copolymer, sodium tauride acrylate/acrylic acid/acrylonitrogen copolymer, solanum tuberosum (potato) starch, starch/acrylates/acrylamide copolymer, starch hydroxypropyltrimonium chloride, steareth-60 cetyl ether, steareth-100/PEG-136/HDI copolymer, sterculia urens gum, synthetic fluorphlogopite, tamarindus indica seed gum, tapioca starch, TEA alginate, TEA carbomer, triticum vulgare (wheat) starch, tromethamine acrylics/acrylonitrogen copolymer, tromethamine magnesium aluminum silicate, welan gum, yeast beta glucan, yeast polysaccharides, zea mays (corn) starch.

Particular preference is given to the polymeric thickener selected from polymeric, anionic, amphiphilic thickeners, particularly preferably those having the INCI names acrylates/beheneth-25 methacrylate copolymer, acrylates/c10-30 alkyl acrylates crosspolymer, acrylates/ceteth-20 itaconates copolymer, acrylates/ceteth-20 methacrylate copolymer, acrylate/laureth-25 methacrylate copolymer, acrylate/palmeth-20 acrylate copolymer, acrylate/palmeth-25 acrylate copolymer, acrylate/palmeth-25 itaconate copolymer, acrylate/steareth-50 acrylate copolymer, acrylate/steareth-20 itaconate copolymer, acrylate/steareth-20 methacrylate copolymer, acrylate/stearyl methacrylate copolymer, acrylate/vinyl isodecanoate crosspolymer, acrylate/steareth-20 methacrylate crosspolymer.

Preferably, the thickening agent or gelling agent is selected from polyacrylic acid, carboxymethyl cellulose, silica, a copolymer of methacrylic acid and methyl methacrylate, and a combination thereof.

Very particular preference is given to the combination of carboxmethylcellulose (for example, Cekol® 50000 ex CP Kelco (INCI: cellulose gum)), a methacrylic acid/methylmethacrylate copolymer (for example Rohagit® S hv ex Evonik (INCI: acrylate copolymer) and silica.

The polymeric thickening agents and gelling agents are preferably contained in the preparation (A) in an amount of from about 0.5 to about 20% by weight, in particular from about 1.0 to about 10% by weight.

The selection of the amount and chemical nature of the thickening agents and gelling agents not only influences the viscosity of the application mixture from preparation (A) and (B) but also the dissolution properties of the preparations (A) and (B). The preferred thickening agents/gelling agents mentioned above also have a particularly advantageous effect on the dissolution properties of the preparations (A) and (B).

However, it has proved to be particularly advantageous to adjust the viscosity of the application mixture obtainable by mixing the preparations (A) and (B) through the selection of a suitable polymer mixture for the water-soluble film. The viscosity of the application mixture and its application properties and bleaching effect can thereby be advantageously influenced both by the chemical nature of the polymer mixture and by the amount of the polymer mixture used for the packaging. Preferred cosmetic agents are therefore exemplified in that the proportion by weight of the polymer mixture with the multi-modal molecular weight distribution to the total weight of preparations (A) and (B) including the water-soluble film is from about 1 to about 15% by weight, preferably from about 2 to about 10% by weight and in particular from about 3 to about 8% by weight.

In addition, the preparation (B) can contain further active ingredients, auxiliaries and additives, for example

nonionic polymers such as, for example, vinylpyrrolidone/vinyl acrylate copolymers, polyvinylpyrrolidone and vinylpyrrolidone/vinyl acetate copolymers and polysiloxanes,

cationic polymers such as quaternized cellulose ethers, polysiloxanes having quaternary groups, dimethyldiallylammonium chloride polymers, acrylamide-dimethyldiallylammonium chloride copolymers, dimethylaminoethyl methacrylate-vinylpyrrolidone copolymers quaternized with diethyl sulfate, vinylpyrrolidone-imidazolinium-methochloride copolymers and quaternized polyvinyl alcohol,

zwitterionic and amphoteric polymers such as, for example, acrylamidopropyltrimethylammonium chloride/acrylate copolymers and octylacrylamide/methyl methacrylate/tert-butylaminoethyl methacrylate/2-hydroxypropylmethacrylate copolymers,

anionic polymers such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic acid anhydride copolymers, and acrylic acid/ethyl acrylate/N-tert-butyl acrylamide terpolymers,

solvents and mediators such as ethanol, isopropanol, ethylene glycol, propylene glycol, glycerin and diethylene glycol,

consistency regulators such as sugar esters, polyol esters or polyol alkyl ethers,

stabilizing agents for hydrogen peroxide, for example complexing agents such as EDTA, NTA, alaninediacetic acid and phosphonic acids,

The composition of some particularly preferred cosmetic preparations as contemplated herein can be found in the following tables (specified in % by weight, based on the total weight of preparations (A) and (B), unless otherwise stated.

| Formula | Formula | Formula | Formula |
|---|---|---|---|
| 1 | 2 | 3 | 4 |

Preparation (A)

| | | | | |
|---|---|---|---|---|
| Peroxodisulfate | 10 to 90 | 20 to 80 | 20 to 80 | 30 to 60 |
| Sodium disilicate | 10 to 50 | 10 to 50 | 10 to 50 | 10 to 50 |
| Other ingredients | ad 100 | ad 100 | ad 100 | ad 100 |

Preparation (B)

| Hydrogen peroxide, | 50 to 98 | 60 to 95 | 80 to 95 | 80 to 95 |
|---|---|---|---|---|
| calculated as | | | | |
| 100% H₂O₂ | | | | |
| Water | 0.5 to 20 | 1.0 to 15 | 1.0 to 15 | 2.0 to 12 |
| Other ingredients | ad 100 | ad 100 | ad 100 | ad 100 |
| | Formula | Formula | Formula | Formula |
| | 6 | 7 | 8 | 9 |

Preparation (A)

| | | | | |
|---|---|---|---|---|
| Peroxodisulfate | 10 to 90 | 20 to 80 | 20 to 80 | 30 to 60 |
| Sodium disilicate | 10 to 50 | 10 to 50 | 10 to 50 | 10 to 50 |
| Thickener and/or | 0.5 to 20 | 0.7 to 15 | 1.0 to 12 | 1.0 to 10 |
| gelling agent | | | | |
| Other ingredients | ad 100 | ad 100 | ad 100 | ad 100 |

Preparation (B)

| Hydrogen peroxide, | 50 to 98 | 60 to 95 | 80 to 95 | 80 to 95 |
|---|---|---|---|---|
| calculated as | | | | |
| 100% H₂O₂ | | | | |
| Water | 0.5 to 20 | 1.0 to 15 | 1.0 to 15 | 2.0 to 12 |
| Other ingredients | ad 100 | ad 100 | ad 100 | ad 100 |
| | Formula | Formula | Formula | Formula |
| | 11 | 12 | 13 | 14 |

Preparation (A)

| | | | | |
|---|---|---|---|---|
| Peroxodisulfate | 10 to 90 | 20 to 80 | 20 to 80 | 30 to 60 |
| Sodium disilicate | 10 to 50 | 10 to 50 | 10 to 50 | 10 to 50 |
| Methacrylic acid/ | 0.5 to 20 | 0.7 to 15 | 1.0 to 12 | 1.0 to 10 |
| methyl methacrylate | | | | |
| copolymer, | | | | |
| carboxymethyl- | | | | |
| cellulose and/or | | | | |
| silica | | | | |
| Other ingredients | ad 100 | ad 100 | ad 100 | ad 100 |

Preparation (B)

| Hydrogen peroxide, | 50 to 98 | 60 to 95 | 80 to 95 | 80 to 95 |
|---|---|---|---|---|
| calculated as | | | | |
| 100% H₂O₂ | | | | |
| Water | 0.5 to 20 | 1.0 to 15 | 1.0 to 15 | 2.0 to 12 |
| Other ingredients | ad 100 | ad 100 | ad 100 | ad 100 |
| | Formula | Formula | Formula | Formula |
| | 16 | 17 | 18 | 19 |

Preparation (A)

| | | | | |
|---|---|---|---|---|
| Peroxodisulfate | 10 to 90 | 20 to 80 | 20 to 80 | 30 to 60 |
| Sodium disilicate | 10 to 50 | 10 to 50 | 10 to 50 | 10 to 50 |
| Methacrylic acid/ | 0.5 to 20 | 0.7 to 15 | 1.0 to 12 | 1.0 to 10 |
| methyl methacrylate | | | | |
| copolymer and | | | | |
| carboxymethyl- | | | | |
| cellulose and silica | | | | |
| Other ingredients | ad 100 | ad 100 | ad 100 | ad 100 |

Preparation (B)

| | | | | |
|---|---|---|---|---|
| Hydrogen peroxide, | 50 to 98 | 60 to 95 | 80 to 95 | 80 to 95 |
| calculated as | | | | |
| 100% H₂O₂ | | | | |
| Water | 0.5 to 20 | 1.0 to 15 | 1.0 to 15 | 2.0 to 12 |
| Other ingredients | ad 100 | ad 100 | ad 100 | ad 100 |

For the application properties of cosmetic agents as contemplated herein, it has proved to be advantageous to reduce as far as possible the proportion by weight of hydrophobic constituents in preparation (B). Preferred cosmetic agents are therefore exemplified in that preparation (B), based on its weight, contains less than 20% by weight, preferably less than 10% by weight and in particular less than 5.0% by weight, of fatty substances. As contemplated herein, the group of the fatty substances is those compounds which are soluble to less than 1 g in 100 g of water at 20° C. These include, for example, waxes such as candelilla wax, carnauba wax or beeswax, shea butter, coconut fat, C₁₂ to C₂₀ fatty acids (in particular palmitic acid, stearic acid), silicones and paraffins.

As stated at the outset, cosmetic agents as contemplated herein are particularly suitable for the preparation of hair-bleaching preparations. A further object of the present application is therefore a method for the color change of keratin fibers, in the course of which

i) at least one packaged preparation (A) and a preparation (B) are mixed into an application mixture in which the preparation (A) packaged in a water-soluble film is introduced into the free-flowing preparation (B) by dissolving the water-soluble film, wherein the preparation (A), based on its own weight, contains
a) from about 10 to about 90% by weight of peroxydisulfate(s)
b) from about 10 to about 50% by weight of sodium silicate and is packaged in a water-soluble film which includes, based on its own weight, at least about 60% by weight, of a polymer mixture that has a multi-modal molecular weight distribution, and wherein the preparation (B) contains water and at least one oxidation agent, exemplified in that the sodium silicate comprises sodium disilicate;
ii) the resulting application mixture is applied to the keratin fibers.

With regard to preferred embodiments of methods as contemplated herein, the said statements are made mutatis mutandis regarding the cosmetic compositions as contemplated herein. In order to avoid repetitions, reference is made at this point to the statements made there.

### EXAMPLES

The following were prepared by mixing their ingredients (specified in % by weight):

| | |
|---|---|
| Preparation (A) | |
| Ammonium persulfate | 10.0 |
| Potassium persulfate | 30.0 |
| Sodium disilicate | 39.4 |
| Methyl methacrylate-methacrylic acid | 1.0 |
| copolymer | |
| Carboxymethylcellulose | 2.0 |
| Silicic acid | 1.0 |
| Magnesium carbonate | 14.7 |
| Polyquaternium-4 | 0.3 |
| EDTA | 1.6 |
| Misc | ad 100 |
| Preparation (B) | |
| Hydrogen peroxide (50% in H₂O) | 12.0 |
| Aculyn ® 33 (28% in H₂O) | 15.0 |
| Sodium lauryl ether sulfate | 2.0 |
| HEDP | 1.5 |
| Sodium hydroxide | 0.76 |
| Water, Misc | ad 100 |

Ingredients:

Aculyn® 33 (ISP Global): Acrylate copolymer

The preparation (A) was packaged in a water-soluble film from Monosol, type LX 9643. The resulting portion pouch was mixed with preparation B in the ratio 1:2. The portion pack dissolves in a few seconds.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for lightening keratin fibers, comprising at least one packaged preparation (A), wherein the preparation (A), based on its total weight, comprises:
a) from about 10 to about 90% by weight of peroxodisulfate(s)
b) from about 10 to about 50% by weight of sodium silicate, wherein the sodium silicate comprises sodium disilicate
and the preparation (A) is packaged in a water-soluble film which, based on its total weight, comprises at least about 60% by weight of a polymer mixture which has a multi-modal molecular weight distribution, wherein a frequency distribution of the molecular weight of the polymer mixture has two or more maximas.

2. The cosmetic agent according to claim 1, wherein the composition (A), based on its total weight, comprises from about 20 to about 80% by weight of peroxodisulfate(s).

3. The cosmetic agent according to claim 1, wherein the sodium silicate, based on its total weight, comprises at least about 20% by weight of sodium disilicate.

4. A cosmetic agent comprising at least one packaged preparation (A), wherein the preparation (A), based on its total weight, comprises:
a) from about 10 to about 90% by weight of peroxodisulfate(s)
b) from about 10 to about 50% by weight of sodium disilicate,
and the preparation (A) is packaged in a water-soluble film which, based on its total weight, comprises at least about 60% by weight of a polymer mixture which has a multi-modal molecular weight distribution, wherein the multi-modal molecular weight distribution means a frequency distribution of the molecular weight has two or more maximas.

5. The cosmetic agent according to claim 4, wherein the water-soluble film, based on its total weight, comprises at least about 95% by weight of a polymer mixture which has a multi-modal molecular weight distribution.

6. The cosmetic agent according to claim 4, wherein the polymer mixture comprises, based on its total weight, at least about 60% by weight of a mixture comprising:
a1) water-soluble vinyl alcohol/vinyl acetate copolymer a1) and
a2) at least one water-soluble vinyl alcohol/vinyl acetate copolymer a2) which is different from the water-soluble vinyl alcohol/vinyl acetate copolymer a1).

7. The cosmetic agent according to claim 4, wherein the polymer mixture comprises, based on its total weight, at least about 60% by weight of a mixture comprising:
a1) water-soluble vinyl alcohol/vinyl acetate copolymer a1) and
a2) at least one optionally modified water-soluble polysaccharide selected from the group of methylcellulose, carboxymethylcellulose, ethylcellulose, hydroxyethylcellulose, hydroxypropylcellulose, hydroxypropylmethylcellulose, dextrin and hydroxypropylstarch.

8. The cosmetic agent according to claim 4, wherein the polymer mixture has a polydispersity index above about 2.2.

9. The cosmetic agent according to claim 4, wherein the cosmetic agent further comprises a flowable preparation (B) which, based on its weight, comprises from about 50 to about 98% by weight of water and from about 0.5 to about 20% by weight of hydrogen peroxide, calculated as 100% H2O2.

10. A method for the color change of keratin fibers, comprising:
i) mixing at least one packaged preparation (A) and a preparation (B) into an application mixture in which the preparation (A), which is packaged in a water-soluble film, is introduced into the free-flowing preparation (B) by dissolving the water-soluble film, wherein the preparation (A), based on its own weight, comprises;
a) from about 10 to about 90% by weight of peroxodisulfate(s)
b) from about 10 to about 50% by weight of sodium silicate, wherein the sodium silicate comprises sodium disilicate
and wherein preparation (A) comprises, based on its own weight, at least about 60% by weight, of a polymer mixture that has a multi-modal molecular weight distribution, wherein the multi-modal molecular weight distribution means a frequency distribution of the molecular weight has two or more maximas, and wherein the preparation (B) comprises water and at least one oxidation agent; and
ii) applying the resulting application mixture to the keratin fibers.

11. The cosmetic agent according to claim 4, wherein the peroxydisulfate is selected from the group of alkali metal and ammonium peroxydisulfates.

12. The cosmetic agent according to claim 11, wherein the peroxydisulfate comprises potassium peroxydisulfate, and wherein the weight ratio of potassium peroxydisulfate present in the agent to the total amount of peroxydisulfates present in the agent is at least 0.5.

13. The cosmetic agent according to claim 4, wherein the preparation (A), based on its total weight, comprises from about 5 to about 30% by weight of carbonate(s).

14. The cosmetic agent according to claim 13, wherein the preparation (A), based on its total weight comprises from about 5 to about 30% by weight of magnesium carbonate.

15. The cosmetic agent according to claim 6, wherein the vinyl alcohol/vinyl acetate copolymer a1) has a polydispersity index of between 1.8 and 2.3.

16. The Cosmetic agent according to claim 6, wherein the vinyl alcohol/vinyl acetate copolymer a1) has a degree of hydrolysis between about 84% and about 90%.

17. The cosmetic agent according to claim 4, wherein the water-soluble film, based on its total weight, has a water content of from about 3.0 to about 12% by weight.

18. The cosmetic agent according to claim 4, further comprising a flowable preparation (B) which comprises water and at least one oxidizing agent.

19. The cosmetic agent according to claim 18, wherein the weight ratio of the preparation (A) to the preparation (B) is from about 1:1 to about 1:10.

20. The cosmetic agent according to claim 4, wherein the composition (A), based on its total weight, comprises:
from about 30 to about 60% by weight of peroxodisulfate(s) selected from the group of alkali metal and ammonium peroxydisulfates wherein the weight ratio of potassium peroxydisulfate present in the agent to the total amount of peroxydisulfates present in the agent is at least 0.5,
from about 5 to about 30% by weight of carbonate(s);
wherein the cosmetic agent further comprises a flowable preparation (B) which, based on its weight, comprises from about 50 to about 98% by weight of water and from about 0.5 to about 20% by weight of hydrogen peroxide, calculated as 100% H2O2;
wherein the weight ratio of the preparation (A) to the preparation (B) is from about 1:1 to about 1:10.
